(19) United States
(12) Patent Application Publication
(10) Pub. No.: US 2009/0054760 A1
(43) Pub. Date: Feb. 26, 2009

(54) Catheter for Enhanced Image Location Detection

(76) Inventor: Harry B. Burke, Glen Allen, VA (US)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 11/844,684

(22) Filed: Aug. 24, 2007

(51) Int. Cl. A61M 25/095

(52) U.S. Cl. 600/424

(57) ABSTRACT

A catheter is used to assist in imaging, e.g., 3D imaging, an anatomical location in a human or veterinary subject. The catheter includes a plurality of horizontal markings which extend in the longitudinal direction between the distal and proximal ends. The horizontal markings can be made throughout the entire length of the catheter, or at one or more locations. At each location, the horizontal markings will appear as successive sets of markings. Each horizontal marking is made of two markings that are the same length. The two markings are arranged such that identically sized gaps between the ends of the markings are positioned on the front and back side of the catheter. In one embodiment, an image where the source and catheter are precisely aligned will show gaps of succeeding larger or smaller sizes. However, when the source is off center the gap lengths will be smaller or not exist. Particular marking locations can be highlighted by using relatively thinner or thicker markings and/or by spacing the markings progressively closer together or further apart.

### BACKGROUND OF THE INVENTION

1. Field of the Invention

The present invention generally relates to catheters used in medical operations for human and animal subjects, and also relates to medical imaging technologies.

2. Background Description

There are a wide variety of medical imaging techniques used in common practice today. Examples include, but are not limited to, computed tomography (“CT scan” or “CAT scan”) which employs the use x-ray beams rotated around a body party with three dimensional images of body parts including organs being developed from computer processing of several individual slices imaged through the body part; magnetic resonance imaging (MRI) which utilize high powered magnets to align hydrogen atoms in the body and computer processing of radio waves reflected from the hydrogen atoms where different tissues are identified by their reflection patterns (three dimensional imaging being similar to CT scans in that the three dimensional images are assembled from multiple slices); and ultrasound imaging which uses sound waves which are reflected by or pass through a body part to image a body part in three dimensions (3D). These techniques are widely practiced in the hospital and veterinary settings.

Catheters are used in a wide variety of medical procedures. In operation, catheters are inserted into a body lumen such as, but not limited to, the urethra, arteries, veins, intestines, etc. Catheters can be used to deliver medicine, perform surgical procedures, to permit cameras or other tools or implants to be delivered to desires locations, or the like. Examples of catheters which include markings on the sidewalls can be found in U.S. Pat. No. 5,188,596 to Condon and U.S. Patent Publication 2006/0020199 to Stubbs both of which are herein incorporated by reference. In both references, a camera or other device is used to image the surface of the urethra through the sidewall of the catheter. While the markings on the catheters disclosed therein can allow for the identification of the location of an anatomical feature, the use of the markings (see FIG. 12 of Condon and FIG. 1b of Stubbs) are limited to use in locating surface features.

### SUMMARY OF THE INVENTION

According to the invention, a catheter with horizontal markings along a length of the catheter can be used to enhance image location detection and in performing 3D imaging processes which employ radiant wave energy such as ultrasound, MRI imaging, and CT scans. In an exemplary embodiment, the catheter will include a plurality of horizontal markings positioned along at least one length of said catheter between said distal end and said proximal end. Each horizontal marking includes two markings that are the same length, where a first of the two markings extends from said front side to the back side on a first side of the catheter, and where a second of the two markings extends from the front side to the back side on a second side of the catheter. The length of the markings will vary along the successive length of the catheter, but will always be is less than half the perimeter of said catheter. The two markings are positioned so that a front side gap and a back side gap of equal size are respectively positioned in horizontal alignment on the front side and the back side of said catheter. In one embodiment, the successive horizontal markings will have gap sizes which preferably gradually increase or gradually decrease in size along the length of the catheter so as to more easily differentiate one marking from another. Alternatively, the gap sizes for each marking can remain generally constant for successive markings or vary in a random or periodic fashion. The successive horizontal markings on the catheter can be spaced at relatively shorter or longer distances apart along the length of the catheter to further highlight different horizontal markings from one another. Alternatively or in addition, the successive horizontal markings on the catheter can be relatively thicker or thinner than preceding markings to better highlight different horizontal markings. The markings will be made from a material which can be easily detected using radiant wave energy in an imaging operation. For example, the markings may be detected for example, by exposing a body part in which the catheter is inserted in a body lumen to ultrasound, x-rays or radio frequency waves, and by detecting the reflected or transmitted signal from the body part. For proper imaging, the markings need to be made from a material or include a material which can be detected separately from the catheter itself. For example, the markings may be made of a polymeric material such as nylon which will have different absorptive or reflective properties to the catheter itself as well as to the body part in which the catheter is inserted. Alternatively, the markings may be made of a material which includes metal or a metallization (such materials would have enhanced reflective properties). Still alternatively, the markings may be made from or include a ceramic material which possesses different absorptive or transmissive properties relative to the catheter. In some applications, the markings and catheter may both be polymeric in character; however, the thickness, choice of polymer, or the incorporation of metal or ceramic particles into the markings can be used to differentiate the markings from the catheter. The markings could be made with an ink which includes polymeric, ceramic, or metallic particles. The principal requirement is that the markings need to be detectable in conjunction with the tissue or organs at or near the body lumen in which the catheter is inserted.

Imaged radiant wave energy that is either transmitted through the body part or reflected by structures in the body part will show the markings on the catheter. Given the three dimensional characteristics of the marking system which include varying gap sizes in combination with either or both varying marking thicknesses and varying spacings of markings, three dimensional analysis preferably using computer processing can be used to determine accurate information related to anatomic location and the location of the transducer.

The catheter has particular utility for use in the urethra for imaging a patient's prostate. However, the catheter can be used in a wide variety of other medical or veterinary settings.

### BRIEF DESCRIPTION OF THE DRAWINGS

The foregoing and other objects, aspects and advantages will be better understood from the following detailed description of a preferred embodiment of the invention with reference to the drawings, in which:

FIGS. 1a and 1b show the front and back side of a catheter;

FIGS. 2a and 2b show alternative exemplary coronal views of the catheter of FIGS. 1a and 1b;

FIG. 3 shows a schematic image of a catheter from a front side view;

FIGS. 4a and 4b show an alternative embodiment where markings on the catheter are of varying thicknesses;

FIGS. 5a and 5b show the front and back sides of an alternative embodiment of the catheter;

FIGS. 6a and 6b show the front and back sides of another alternative embodiment of the catheter; and

FIG. 7 is a schematic view showing imaging of a prostate where a balloon catheter, marked as shown in FIGS. 1a-b or 4a-b is inserted in the urethra of a human patient.

### DETAILED DESCRIPTION OF THE PREFERRED EMBODIMENTS OF THE INVENTION

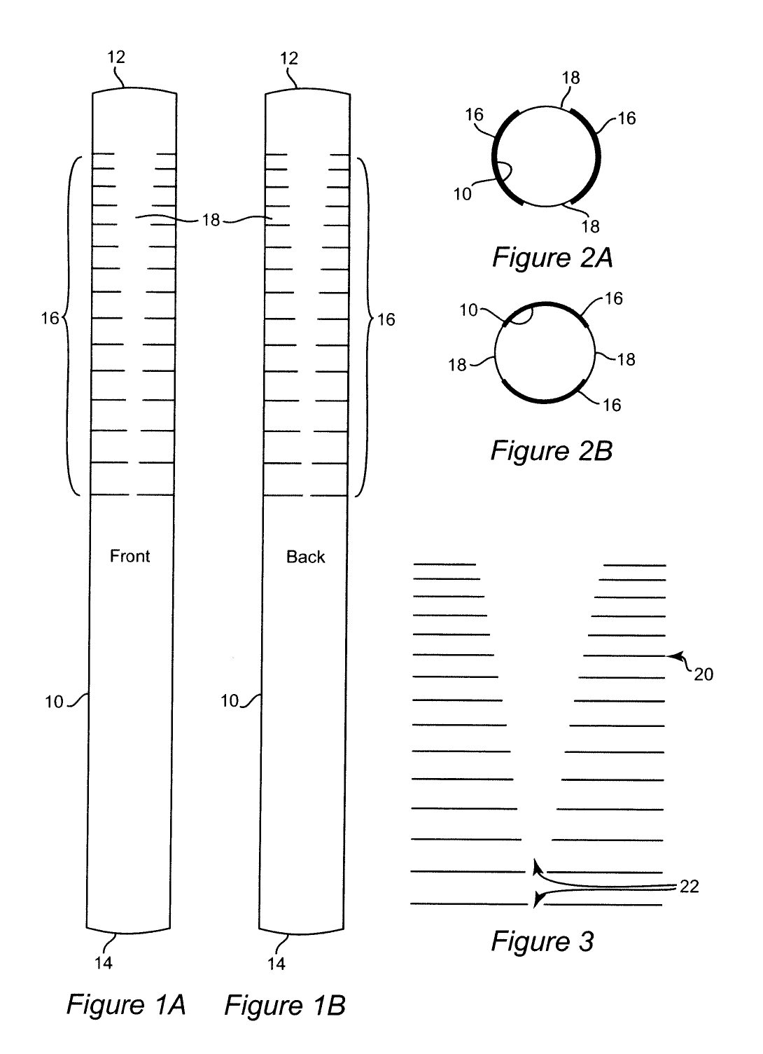

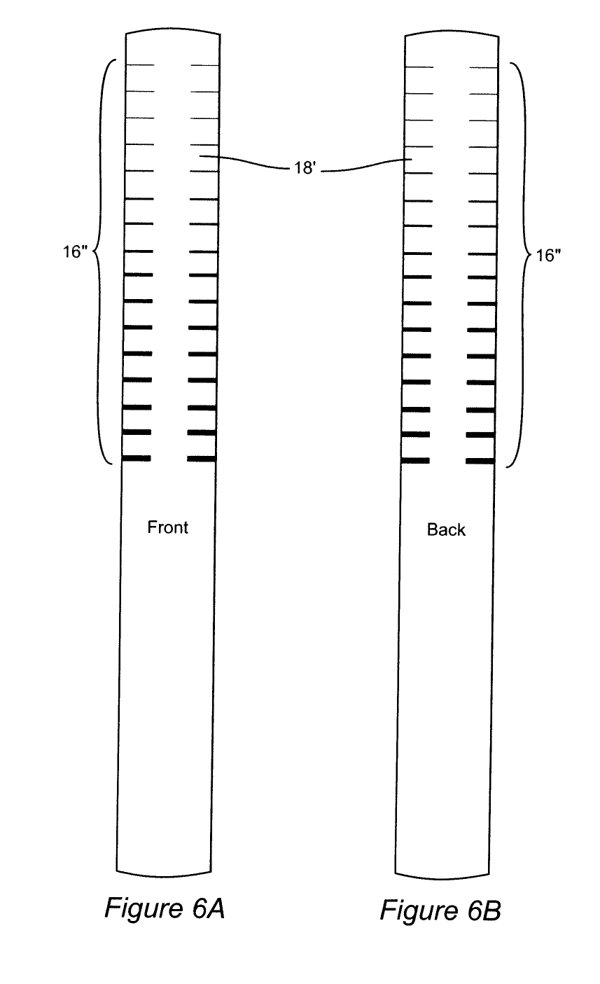

FIGS. 1a and 1b respectively show the front and back side of a catheter 10 having a proximal end 12 and distal end 14. Horizontal markings 16 are shown on the catheter 10 towards the proximal end 12. The horizontal markings 16 each have a gap 18 on the front surface (FIG. 1a) and back surface (FIG. 1b) which are in alignment with a diameter through the front and back surfaces. For exemplary purposes, the gap 18 is shown to be progressively larger towards the proximal end 12 of the catheter 10; however, it should be understood that the gap 18 could be progressively smaller towards the proximal end 12. Contrasting the catheter of FIG. 1a with the catheter of FIGS. 6a and 6b, in some applications the gap 18′ may be constant or may change in some other fashion than becoming progressively larger or smaller. With further reference to FIGS. 1a and 1b, in one embodiment the horizontal markings 16 are progressively closer together towards the proximal end in terms of the lengthwise or longitudinal direction of the catheter 10 for successive horizontal markings 16. However, the longitudinal spacing of horizontal markings 16 could progressively become closer together towards the distal end in another configuration.

FIGS. 1a and 1b show the horizontal markings on at least one length of the catheter 10. However, it should be understood that the horizontal markings 16 could be positioned on the entire length of the catheter 10. In addition, the horizontal markings 16 could be positioned on two or more locations of the same catheter 10.

FIGS. 2a and 2b are plan views of the catheter 10 which illustrate a cross-sectional view through the catheter 10 which will be discussed in more detail in conjunction with FIG. 3.

FIG. 2a illustrates one embodiment where the horizontal markings 16 are positioned on an external surface of the catheter 10. In contrast, FIG. 2b illustrates an alternative embodiment where the horizontal markings 16 are positioned on an internal surface of the catheter 10. It is also possible that the horizontal markings themselves can be formed in the material which makes the catheter 10. The choice of material for the horizontal markings 16 can vary widely within the practice of the invention with the chief purpose being that the markings 16 can be detected using the radiant wave energy of choice (e.g., ultrasound, x-rays, rf waves, etc.). For example, the horizontal markings 16 could be made of a polymeric material such as nylon which would be more absorptive and less reflective. Alternatively, the horizontal markings 16 could be made of a metallic material or metallized polymer which would be more reflective. As a further alternative, the horizontal markings 16 could be made of a ceramic material or contain ceramic particulate material. The chief requirement is the markings 16 be discernable from the material which makes the catheter 10 (e.g., in some applications, an encapsulated liquid might function as a horizontal marking). Further, the thickness of the horizontal markings both in the surface area dimension and in the dimension through the marking itself will depend on the type of radiant wave energy used and the environment in which the catheter 10 will be used.

FIGS. 2a and 2b also highlight the horizontal markings 16 being constructed from two marks of identical lengths. The marks span less than 180 degrees around the catheter 10, on first and second sides of the catheter 10, so as to leave gaps 18. The gaps 18 on the front and back side of the catheter 10 are of identical dimensions and are in alignment. While catheter 10 is shown as being hollow, it could also be solid. Further catheter 10 might also be polygonal in character instead of cylindrical.

With reference to FIGS. 1a, 1b, 2a, and 3, it can be seen that if a source (not shown) is positioned directly in front of the catheter 10 (view from the top of FIG. 2a), the image of the catheter (FIG. 3), which can be either transmitted or reflected will produce a pattern 20 identical to the horizontal markings 16 on the catheter 10. In the embodiment shown, the gaps 18 progressively increase towards the proximal end 12 of the catheter (FIG. 1a), and this results in a centrally located V shape in the imaged pattern 20 of FIG. 3.

However, if the source was positioned directly in front of the catheter 10 (cross-sectional view from the top of FIG. 2b), the horizontal markings 16 would all be directly in front of the source, and the image would be simply straight lines, as no gaps would be detected. In this case, if one were imaging a patient, one could rotate the catheter 10 while it is in the lumen of a body cavity so that the gaps 18 would be more aligned with the source and would show up on the imaged pattern 20 (FIG. 3).

Often, the source will not be aligned perfectly with the catheter 10. The gaps 18 help to locate the source during imaging. For example, with reference to FIG. 3, if the source were slightly off center, the bottom one or more lines of the imaged pattern 20 would have no opening 22, and the openings 22 would all be smaller towards the top of the pattern 20 (relative to a straight on association of source to catheter). This is because the offset of the source from a straight on view through the smallest gaps 18 results in radiant wave energy not being able to pass directly through the catheter 10. Thus, one can more easily locate the source by analyzing the changes in the pattern 20 and optimizing the pattern 20 to include openings 22 for every horizontal marking. Alternatively, based on the pattern itself the anatomic location of an image or the source might be computed. Thus, the catheter 10 will enable the enhanced construction of 3D images where the anatomic locations are precisely known.

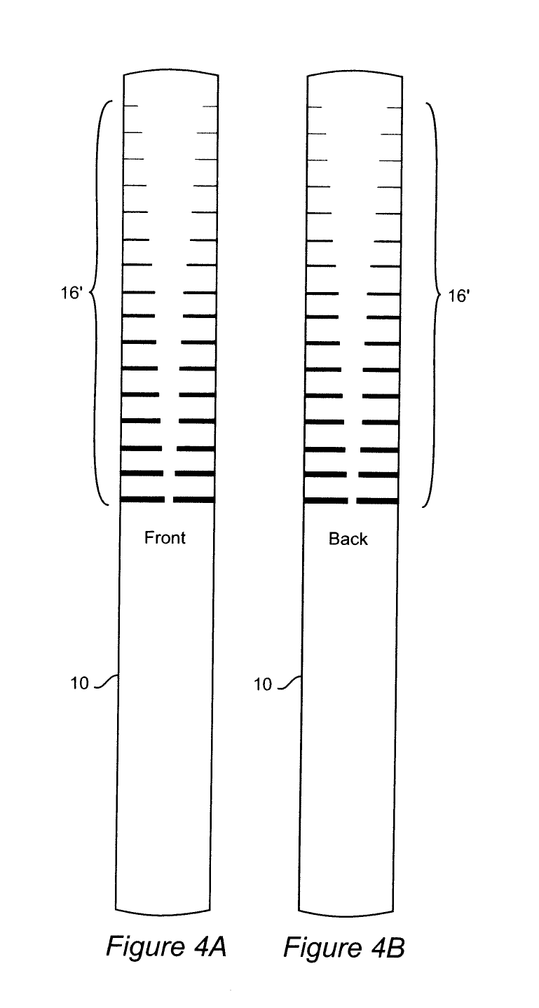

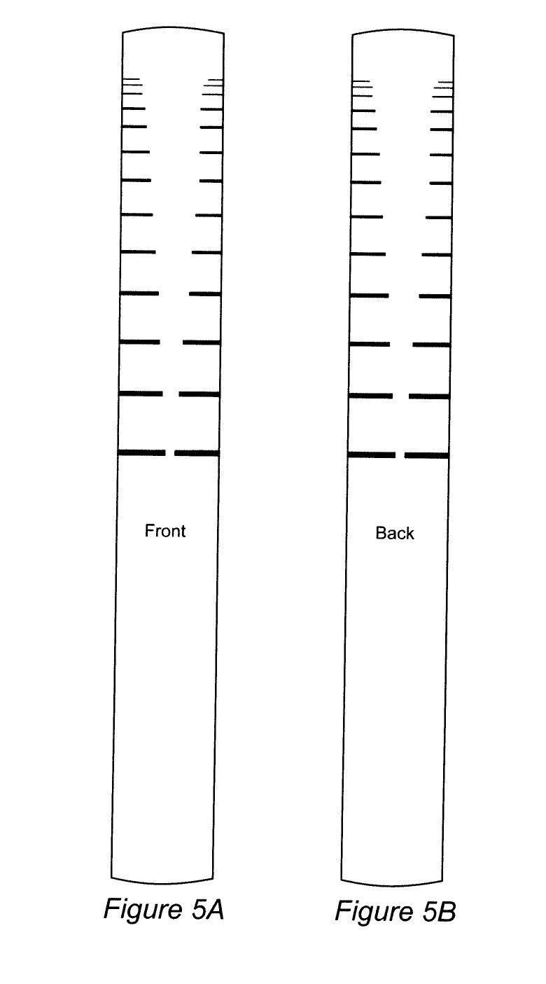

While FIGS. 1a and 1b show the ability to highlight specific horizontal markings 16 for identification purposes by varying the longitudinal spacing between the markings, FIGS. 4a and 4b show an alternative where the thickness of the horizontal markings 16′ are varied and the spacing between markings is more regular. FIGS. 5a and 5b shows an embodiment where a combination of thickness variation (FIGS. 4a and 4b) and spacing variation (FIGS. 1a and 1b) can be employed to highlight specific horizontal markings 16.

FIGS. 6a and 6b show an alternative embodiment where the gap 18′ between markings 16″ remains constant. This configuration provides for some of the alignment detection benefits discussed above in conjunction with FIGS. 2a, 2b, and 3. It should be understood that the gap 18′ might also vary in some applications in a random or periodic pattern, as opposed to a progressively larger or smaller dimension as shown in FIGS. 1a and 1b.

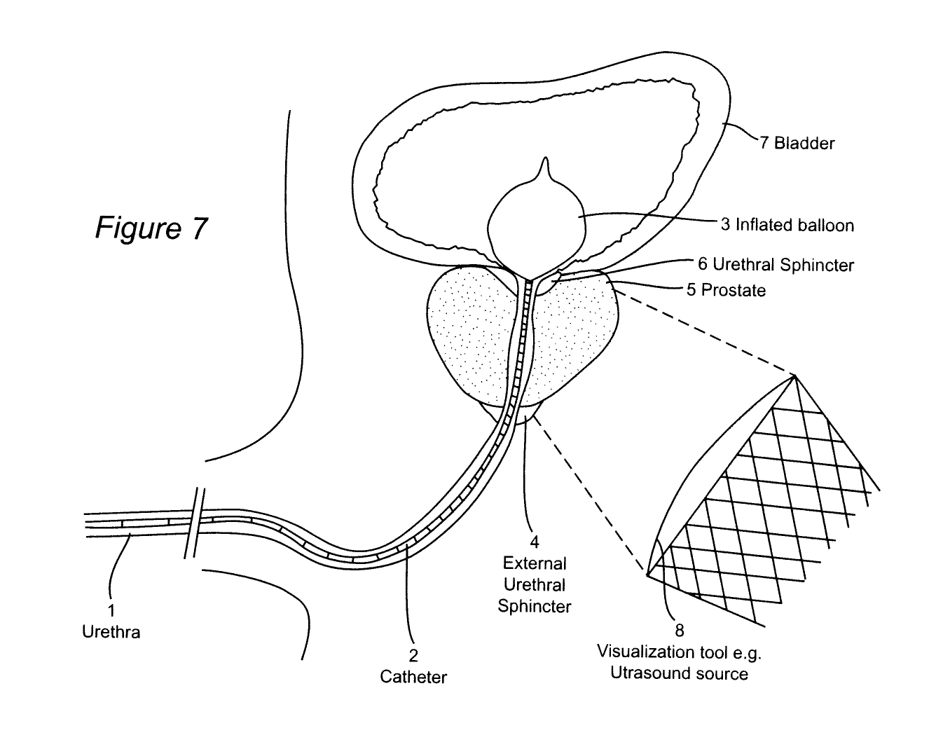

FIG. 7 illustrates a specific embodiment where the catheter 2 is inserted into the urethra 1 of a male patient. The catheter 2 preferably has an inflatable balloon 3 at one end which serves to anchor the catheter in the patient's bladder 7. The catheter 10 includes horizontal markings along its length and these markings can vary in spacing as is best shown in FIGS. 1a and 1b. While it is not shown in FIG. 5, it should be understood that the front of the catheter and the rear of the catheter have a pattern of horizontal markings 16 and gaps 18 similar to that shown in FIGS. 1a and 1b, and in the case shown in FIG. 5 the front of the catheter is facing the visualization tool 8. The visualization tool 8 can be an ultrasound source, rf source, x-ray source, etc.; however, it is envisioned that ultrasound would be appropriate for a wide variety of applications. While FIG. 5 shows markings the entire length of the catheter 2, the catheter 2 could have different sets of markings at, for example, the urethral sphincter 6, the external urethral sphincter 4, and at the urethra 1 which will provide some assurance that the balloon is anchored in the correct location for imaging purposes, and will allow for comparison of images taken at different times (it being recognized that the catheter 2 anchor location may vary over time).

While the invention has been described in terms of its preferred embodiments, those skilled in the art will recognize that the invention can be practiced with modification within the spirit and scope of the appended claims.

1. A catheter having a front side, a back side, a first side, a second side, a distal end, and a proximal end, said catheter comprising:
a plurality of horizontal markings positioned along at least one length of said catheter between said distal end and said proximal end,
each horizontal marking comprising two markings of a same length, a first of said two markings extending from said front side to said back side on said first side of said catheter, and a second of said two markings extending from said front side to said back side on said second side of said catheter, wherein said same length is less than half the perimeter of said catheter and said two markings are positioned so that a front side gap and a back side gap of equal size are respectively positioned in horizontal alignment on said front side and said back side of said catheter, and
wherein successive horizontal markings of said plurality of horizontal markings have gap sizes which increase or decrease in size along said at least one length of said catheter.

2. The catheter of claim 1 wherein said successive horizontal markings are relatively thicker or thinner than preceding markings along said at least one length of said catheter.

3. The catheter of claim 1 wherein said successive horizontal markings are spaced at relatively shorter or longer distances apart along said at least one length of said catheter.

4. The catheter of claim 1 wherein said plurality of horizontal markings are made of a material has properties that differ from said catheter relative to radiant wave energy.

5. The catheter of claim 4 wherein said material is polymeric.

6. The catheter of claim 4 wherein said material is or contains a metallic component.

7. The catheter of claim 4 wherein said material is or contains a ceramic component.

8. The catheter of claim 1 wherein said radiant wave energy is selected from ultrasound, radio frequency, and x-rays.

9. The catheter of claim 1 wherein said horizontal markings are positioned on an external surface of said catheter.

10. The catheter of claim 1 wherein said horizontal markings are positioned on an internal surface of said catheter.

11. The catheter of claim 1 wherein said horizontal markings are formed in a surface of said catheter.

12. The catheter of claim 1 wherein said plurality of horizontal markings are positioned at least two separate lengths of said catheter.

13. The catheter of claim 1 further comprising a balloon member positioned at said distal end.

14. A catheter having a front side, a back side, a first side, a second side, a distal end, and a proximal end, said catheter comprising:
a plurality of horizontal markings positioned along at least one length of said catheter between said distal end and said proximal end,
each horizontal marking comprising two markings of a same length, a first of said two markings extending from said front side to said back side on said first side of said catheter, and a second of said two markings extending from said front side to said back side on said second side of said catheter, wherein said same length is less than half the perimeter of said catheter and said two markings are positioned so that a front side gap and a back side gap of equal size are respectively positioned in horizontal alignment on said front side and said back side of said catheter, and
wherein at least one of (i) or (ii) are satisfied
(i) wherein said successive horizontal markings are relatively thicker or thinner than preceding markings along said at least one length of said catheter,
(ii) wherein said successive horizontal markings are spaced at relatively shorter or longer distances apart along said at least one length of said catheter.

15. The catheter of claim 14 wherein said plurality of horizontal markings are made of a material has properties that differ from said catheter relative to radiant wave energy.

16. The catheter of claim 15 wherein said material is polymeric.

17. The catheter of claim 15 wherein said material is or contains a metallic component.

18. The catheter of claim 15 wherein said material is or contains a ceramic component.

19. The catheter of claim 14 wherein said radiant wave energy is selected from ultrasound, radio frequency, and x-rays.

20. The catheter of claim 14 wherein said horizontal markings are positioned on an external surface of said catheter.

21. The catheter of claim 14 wherein said horizontal markings are positioned on an internal surface of said catheter.

22. The catheter of claim 14 wherein said horizontal markings are formed in a surface of said catheter.

23. The catheter of claim 14 wherein said plurality of horizontal markings are positioned at least two separate lengths of said catheter.

24. The catheter of claim 14 further comprising a balloon member positioned at said distal end.
